(19) United States
(12) Patent Application Publication
(10) Pub. No.: US 2019/0117432 A1
(43) Pub. Date: Apr. 25, 2019

(54) THREE-DIMENSIONAL BODY ORTHOPRAXIS APPARATUS

(71) Applicant: Yoon Mi PARK, Gyeonggi-do (KR)

(72) Inventor: Yoon Mi PARK, Gyeonggi-do (KR)

(21) Appl. No.: 15/743,187

(22) PCT Filed: Jul. 13, 2016
(86) PCT No.: PCT/KR2016/007584

§ 371 Date: Jan. 9, 2018

(30) Foreign Application Priority Data
Jul. 14, 2015 (KR) 10-2015-0099604

(51) Int. Cl. A61F 5/02

(52) U.S. Cl. CPC A61F 5/026; A61F 5/028; A61F 5/024

(57) ABSTRACT

A three-dimensional body orthopraxis apparatus includes: a top point support portion which supports a top point displaced relative to a normal state of both left and right ends of a body; an tilt belt portion which is obliquely extended downward from the top point support portion and exerts tension in the opposite direction of a rotational malposition of the deformed top point with respect to the top point support portion; a horizontal belt portion which surrounds the body in a horizontal direction and pressurizes the body; at least one cushion pad which is pressed towards the inside of the body by means of at least a part of the horizontal belt portion to pressurize a part of the body; and a flexible skin which is disposed inside the tilt belt portion and the horizontal belt portion.

### TECHNICAL FIELD

The present invention relates to a body orthopraxis apparatus which keeps an inherent or acquired shape deformation of a body, and more particularly, to a belt type orthopraxis apparatus adequate for orthopraxis of scoliosis.

### BACKGROUND ART

Scoliosis is an abnormal deformity of the spine and generally refers to the spine being bent sideways in a front view. However, actually, scoliosis is not a simple two-dimensional deformity but is accompanied by a rotational distortion of a vertebral body such that, when viewed from the side, the spine is in a three-dimensionally deformed state rather than a normally curved state.

FIG. 1 illustrates a skeletal shape of a scoliosis patient. As shown, when viewed from the patient's back, a thoracic vertebral area is bent rightward. A central portion of the thoracic vertebrae is turned clockwise relative to a direction of looking down on the patient from above such that the right costa protrudes rearward. However, the spine is distorted counterclockwise toward a cervical spine side such that the right shoulder is relatively raised upward and simultaneously faces frontward. Also, the pelvis is distorted clockwise such that the left ilium is raised upward and simultaneously faces frontward.

FIG. 2 is a mimetic diagram illustrating deformities of a body of a scoliosis patient. Like FIG. 1, a central part of a central line SP of the spine, that is, a thoracic vertebral area, is curved rightward. As a result thereof, a right side of a shoulder line SH is raised upward and simultaneously distorted counterclockwise, and an inner part of the right scapula protrudes rearward. A right side of a chest line CH is raised upward and simultaneously distorted clockwise, and a left side of a pelvic line PE is slightly high and distorted clockwise. As described above, deformities of body according to scoliosis occur in three-dimensions. Also, due to a rotational distortion of the spine, a right side of the costae protrudes rearward and a left side thereof protrudes frontward such that a rear side of the left side is depressed inward. The deformities tend to proceed continuously such that, when deformities of the thoracic are worsen, functions of the internal organs are degraded such that lung capacity is reduced and dyspnoea occurs.

In the field of medial auxiliary apparatuses, a variety of orthopraxis apparatuses have been provided to reduce the inconvenience and danger caused by scoliosis. For example, there are Korean Patent Publication No. 10-2010-89953 titled “Scoliosis Orthosis,” Korean Utility Model Publication No. 20-2010-5951 titled “Orthopraxis Belt for Body Orthopraxis,” and the like, which have complicated structures, are difficult to wear, and are incapable of realigning three-dimensional deformities including a rotational distortion of the spine. Meanwhile, as one method of solving the problems, the present applicant has filed “Belt Type Scoliosis Orthopraxis Apparatus” disclosed in Korean Patent Registration Publication No. 10-1508579.

### DISCLOSURE OF INVENTION

### Technical Problem

Therefore, it is an aspect of the present invention to provide a three-dimensional body orthopraxis belt capable of realigning not only two-dimensional deformities in which a central line of the spine is bent sideways but also three-dimensional deformities which accompany a turn of the spine.

Also, it is another aspect of the present invention to provide a three-dimensional body orthopraxis belt capable of effectively realigning deformities of a thoracic vertebral area including the shoulders and costae as well as deformities of the back, pelvis, and coxa areas.

### Technical Solution

According to one aspect of the present invention, a three-dimensional body orthopraxis apparatus includes a top point support portion which supports a top point which is in a malpositioned state rather than a normal state of both left and right ends of a body a tilt belt portion which at least partially includes an elastic belt, obliquely extends downward from the top point support portion, and applies tension to the top point support portion in a direction opposite a rotational malposition of the top point, a horizontal belt portion which at least partially includes an elastic belt and horizontally surrounds and pressurizes the body to partially overlap a bottom end of the tilt belt portion, and an overlap support portion which forms a boundary at which the bottom end of the tilt belt portion and the horizontal belt portion meet, and is pressurized toward an inside of the body by at least a part of the horizontal belt portion to be supported not to slide on the body. Here, the top point support portion includes an annular shoulder band which annularly surrounds a perimeter of one shoulder, and the annular shoulder band is connected to the tilt belt portion at a rear side of the shoulder.

The top point support portion may further include a scapula pad disposed at a position corresponding to a scapula, which protrudes rearward, and pressurized toward the inside of the body by the tensions of the shoulder band and the tilt belt portion.

The three-dimensional body orthopraxis apparatus may further include a costa pad disposed to overlap the horizontal belt portion, disposed at a position corresponding to a protruding costal area, and pressurized toward the inside of the body by the tension of the horizontal belt portion.

According to another aspect of the present invention, a three-dimensional body orthopraxis apparatus includes a top point support portion which supports a top point which is in a malpositioned state rather than a normal state of both left and right ends of a body a tilt belt portion which at least partially includes an elastic belt, obliquely extends downward from the top point support portion, and applies tension to the top point support portion in a direction opposite a rotational malposition of the top point, a horizontal belt portion which at least partially includes an elastic belt and horizontally surrounds and pressurizes the body to partially overlap a bottom end of the tilt belt portion, and an overlap support portion which forms a boundary at which the bottom end of the tilt belt portion and the horizontal belt portion meet, and is pressurized toward an inside of the body by at least a part of the horizontal belt portion to be supported not to slide on the body. Here, the top point support portion includes a pelvis support portion corresponding to an iliac crest area of one side of the pelvis of a wearer which is relatively raised upward, and may further includes a pair of coxa pads arranged between the horizontal belt portion and the body to correspond to both coxa areas.

According to still another aspect of the present invention, a three-dimensional body orthopraxis apparatus includes a top point support portion which supports a top point which is in a malpositioned state rather than a normal state of both left and right ends of a body a tilt belt portion which at least partially includes an elastic belt, obliquely extends downward from the top point support portion, and applies tension to the top point support portion in a direction opposite a rotational malposition of the top point, a horizontal belt portion which at least partially includes an elastic belt and horizontally surrounds and pressurizes the body, at least one cushion pad which is pressurized toward an inside of a body by at least a part of the horizontal belt portion and pressurizes a part of the body, and a flexible skin which is disposed inside the tilt belt portion and the horizontal belt portion to be in contact with the body of a wearer and defines relative positions of the tilt belt portion, the horizontal belt portion, and the at least one cushion pad.

The at least one cushion pad may be fixed to one position inside the flexible skin, and at least a part of each of the tilt belt portion and the horizontal belt portion may be supported by the flexible skin such that a position thereof may be defined.

An overlap part of the flexible skin which overlaps the horizontal belt portion may surround the malpositioned body part of the wearer, may be formed to allow both ends thereof to be attached to each other, and may be formed to allow the tilt belt portion and the horizontal belt portion to be attached to parts of an outer surface of the flexible skin while tension is applied thereto. The attachment while the tension is applied refers to, for example, a case in which an elastic belt member which forms at least parts of the tilt belt portion and the horizontal belt portion stretches and an end portion thereof is attached to the outer surface of the flexible skin.

The top point support portion may include a top point pressurizing pad which protrudes inward to pressurize a part of the body.

The top point pressurizing pad may be a pelvic pad corresponding to an iliac crest area of one side of the pelvis of the wearer which is relatively raised upward. The at least one cushion pad may be a pair of coxa pads fixed to positions corresponding to both coxa areas of the wearer. The tilt belt portion may include a front tilt belt which extends from the pelvis pad toward a front side of the wearer and a rear tilt belt which extends toward a rear side of the wearer. The front tilt belt and the rear tilt belt may be attached to the outer surface of the flexible skin. Here, an auxiliary belt which extends from both sides of the pelvis pad and is supported by the body part of the wearer to apply a force pulling the pelvis pad downward may be further included.

The top point support portion may include an annular shoulder band which annularly surrounds a perimeter of one frontwardly distorted shoulder, and the annular should band may be connected to the tilt belt portion at a rear side of the shoulder.

The top point support portion may further include a scapula pad disposed at a position corresponding to a scapula, which protrudes rearward, and pressurized toward the inside of the body by the tensions of the annular shoulder band and the tilt belt portion.

The horizontal belt portion may include a front horizontal belt which extends from a cushion pad of the at least one cushion pad which is relatively closer to the top point support portion toward the front side of the wearer, and a rear horizontal belt which extends toward the rear side of the wearer. Also, the front horizontal belt and the rear horizontal belt may be formed to be attached to an outer surface of the flexible skin.

The horizontal belt portion may include a movable pulley type belt tension structure.

According to yet another aspect of the present invention, a three-dimensional body orthopraxis apparatus may include a three-dimensional upper body orthopraxis apparatus and a three-dimensional lower body orthopraxis apparatus. Here, the three-dimensional upper body orthopraxis apparatus may include an annular shoulder band which annularly surrounds a perimeter of one shoulder which is in a malpositioned state rather than a normal state of shoulders of a wearer, a first tilt belt portion which at least partially includes an elastic belt, obliquely extends downward from a rear side of the annular shoulder band, and applies tension in a direction opposite the malposition of the one shoulder, and a first horizontal belt portion which at least partially includes an elastic belt, horizontally surrounds and pressurizes the body of the wearer, and directly or indirectly supports a bottom end of the first tilt belt portion. Also, the three-dimensional lower body orthopraxis apparatus may include a pelvis support portion which supports an iliac crest area of one side of the pelvis of the wearer which is in a malpositioned state rather than a normal state, a second tilt belt portion which obliquely extends downward from both sides of the pelvis support portion and applies tension in a direction opposite the rotational malposition, and a second horizontal belt portion which at least partially includes an elastic belt, horizontally surrounds and pressurizes the body of the wearer, and directly or indirectly supports a bottom end of the second tilt belt portion.

Here, the directly or indirectly supporting, by the first horizontal belt portion and the second horizontal belt portion, the bottom ends of the first tilt belt portion and the second tilt belt portion may include components which support the bottom ends by indirectly transferring a force through another member as well as components which support the bottom ends by coming into direct contact with each other or being connected to each other.

### Advantageous Effects

The three-dimensional body orthopraxis apparatus according to the embodiments of the present invention has a relatively simple and intuitive shape and is capable of realigning three-dimensional deformities including a curve and turn of the spine such that adjustment according to a degree of body deformity is easily performed.

Also, the three-dimensional body orthopraxis apparatus according to the embodiments of the present invention may realign deformities of a thoracic vertebrae area which accompany deformities of the shoulders and costae as well as deformities of the back, pelvis, and coxae. Through this, a phenomenon in which a center of an upper body is shifted due to a shear and distortion between centers of the upper body and a lower body may be prevented and effectively realigned.

### BRIEF DESCRIPTION OF DRAWINGS

FIG. 1 illustrates a skeletal shape of a scoliosis patient.

FIG. 2 is a mimetic diagram illustrating deformities of a body of a scoliosis patient.

FIG. 3 illustrates a state in which a three-dimensional upper body orthopraxis apparatus according to one embodiment of the present invention is worn.

FIG. 4 is a detailed view illustrating components of the three-dimensional upper body orthopraxis apparatus according to the embodiment of FIG. 3.

FIG. 5 is a cross-sectional view taken along V-V′ of FIG. 4.

FIG. 6 is a cross-sectional view taken along VI-VI′ of FIG. 4.

FIG. 7 illustrates a state in which a three-dimensional lower body orthopraxis apparatus according to one embodiment of the present invention is worn.

FIG. 8 is a detailed view illustrating components of the three-dimensional lower body orthopraxis apparatus according to the embodiment of FIG. 7.

FIG. 9 is a partially enlarged view illustrating an overlap support portion in FIG. 8.

FIG. 10 illustrates a state in which a three-dimensional upper body orthopraxis apparatus according to one embodiment of the present invention is worn.

FIG. 11 is a detailed view illustrating components of the three-dimensional upper body orthopraxis apparatus according to the embodiment of FIG. 10.

FIG. 12 illustrates an operation principle of a movable pulley type belt tension structure in the embodiment of FIG. 11.

FIG. 13 illustrates a state in which a three-dimensional lower body orthopraxis apparatus according to one embodiment of the present invention is worn.

FIG. 14 is a detailed view illustrating components of the three-dimensional lower body orthopraxis apparatus according to the embodiment of FIG. 13.

FIG. 15 illustrates an operation principle of a movable pulley type belt tension structure in the embodiment of FIG. 14.

FIG. 16 illustrates a modified example of the embodiment of FIG. 13.

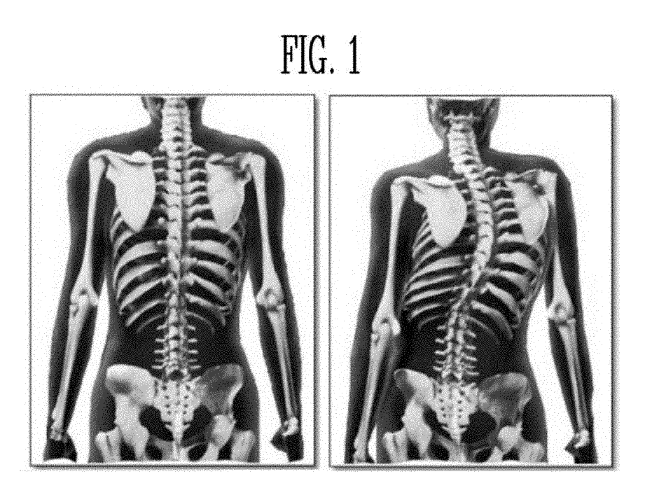

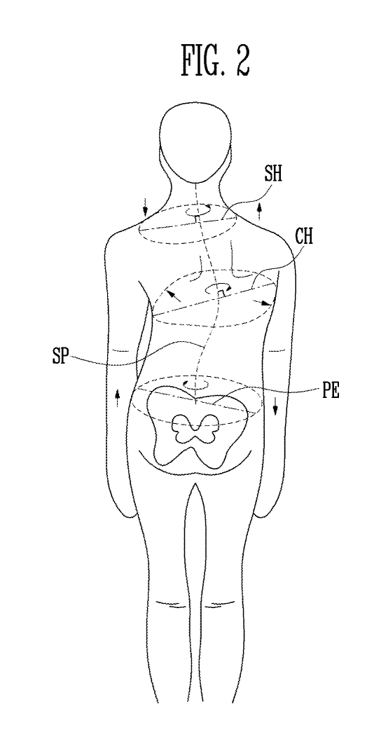

### MODE FOR INVENTION

Hereinafter, embodiments of the present invention will be described with reference to the attached drawings. The embodiments described below may be modified in a variety of shapes, and the scope of the present invention is not limited to the following embodiments. The embodiments of the present invention are provided to clearly transfer the technical concept of the present invention to one of ordinary skill in the art.

A three-dimensional body orthopraxis apparatus may be used for orthopraxis when there is a distorted part which is moved upward from a normal state of left and right ends of a body and is simultaneously rotatably malpositioned frontward and rearward. The three-dimensional body orthopraxis apparatus may be applied to orthopraxis of thoracic deformities including a tilt and distortion of the shoulders and a costal area, orthopraxis of pelvic deformities including a tilt and distortion of pelvic bones connected to the spine, and the like. Hereinafter, for convenience, an embodiment more adequate for the former case will be referred to as a three-dimensional upper body orthopraxis apparatus, and an embodiment more adequate for the latter case will be referred to as a three-dimensional lower body orthopraxis apparatus.

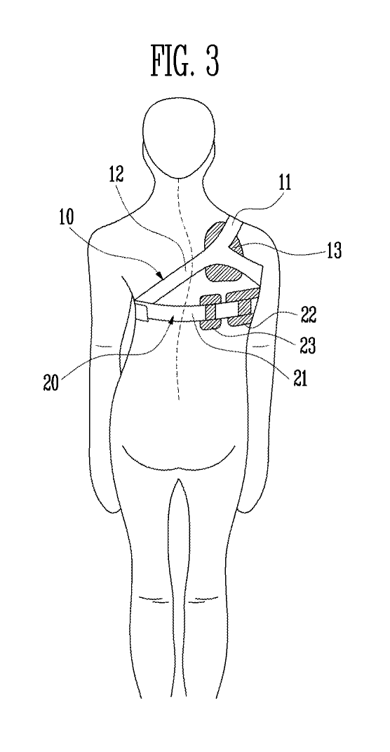

FIG. 3 illustrates a state in which a three-dimensional upper body orthopraxis apparatus according to one embodiment of the present invention is worn. FIG. 4 is a detailed view illustrating components of the three-dimensional upper body orthopraxis apparatus according to the embodiment of FIG. 3.

The three-dimensional upper body orthopraxis apparatus according to the embodiment mainly includes a tilt belt portion 10 and a horizontal belt portion 20. A shoulder band 11 which surrounds one shoulder which is relatively raised upward and distorted frontward of both the shoulders of a wearer is provided at a top end of the tilt belt portion 10. The shoulder band 11 is one example of a top point support portion which supports a top point of left and right ends of the body which is raised higher than a normal state and simultaneously rotatably distorted frontward and rearward, and the shoulder band 11 is connected to the tilt belt 12 which obliquely extends downward at a rear side opposite the distortion of the shoulder. The tilt belt 12 is formed of an elastic belt member having elasticity and applies tension to the shoulder band 11. The shoulder band 11 may also be formed of an elastic belt member. Meanwhile, a scapular pad 13 which pressurizes the scapula which protrudes rearward using tension of an elastic belt member may be provided at a part at which the shoulder band 11 and the tilt belt 12 meet.

A bottom end of the tilt belt 12 is connected to the horizontal belt portion 20, and an overlap support portion which overlaps one part of the horizontal belt portion 20 surrounding the body in a loop shape and pressurizing the body inward and is supported by a force of the pressurization not to slide on the body is formed at a boundary therebetween. The overlap support portion may include, for example, a front costa pad 15. The front costa pad 15 is disposed at a position corresponding to a costal part which protrudes from one front side of the wearer and is approximately located on a diagonally opposite side of the above-described scapular pad 13. One side of the front costa pad 15 is connected to the above-described tilt belt 12, and the other side is connected to a horizontal belt 21.

The tilt belt 12 and the front costa pad 15 are connected by a length adjustor. The length adjustor adjusts tension applied to the tilt belt 12 according to a degree of body deformity of the wearer as well as adjusts a length of the tilt belt 12 according to a body size of the wearer. Since the overlap support portion, which is an example of the front costa pad 15, is supported not to slide on the body of the wearer, the tension applied to the shoulder band 11 is strengthened as the length of the tilt belt 12 is decreased.

The horizontal belt 21, which starts from the front costa pad 15, may surround the thorax of the wearer in a loop shape, may reach and overlap at least a part of an outer surface of the front costa pad 15, and may be fixed to a part of an outer surface of the horizontal belt 21 by a fixing portion 24. The fixing portion 24 may be configured to adjust a length of the part of the horizontal belt 21 which surrounds the body through fixation like, for example, Velcro. However, the fixing portion 24 is not limited thereto, and may be embodied with a variety of methods such as a hook, a buckle, and the like.

Meanwhile, among sections which the horizontal belt 21 passes, a rear costa pad 22 pressurized toward the inside of the body by the tension of the horizontal belt 21 may be provided at a position corresponding to a rear costal area which protrudes from the wearer. Also, when it is necessary to additionally pressurize an area according to a body condition of the wearer, an auxiliary pressure pad 23 may be further provided at a corresponding position. The auxiliary pressure pad 23 may be installed such that a position thereof is movable in a longitudinal direction of the horizontal belt 21.

The horizontal belt portion 20 performs orthopraxis by pressurizing the thorax, which protrudes in a direction in which it is distorted with respect to a lateral axis which connects both ends of the body, toward the inside of the body by using the horizontal belt 21 formed of the elastic belt member. The horizontal belt portion 20 includes the front costa pad 15, the rear costa pad 22, and the like, intensively pressurizes an abnormally protruding part, and prevents a pressure from being applied to a relatively depressed part due to deformities. The inclusion of the front and rear costa pads 15 and 22 will be described in detail as follows.

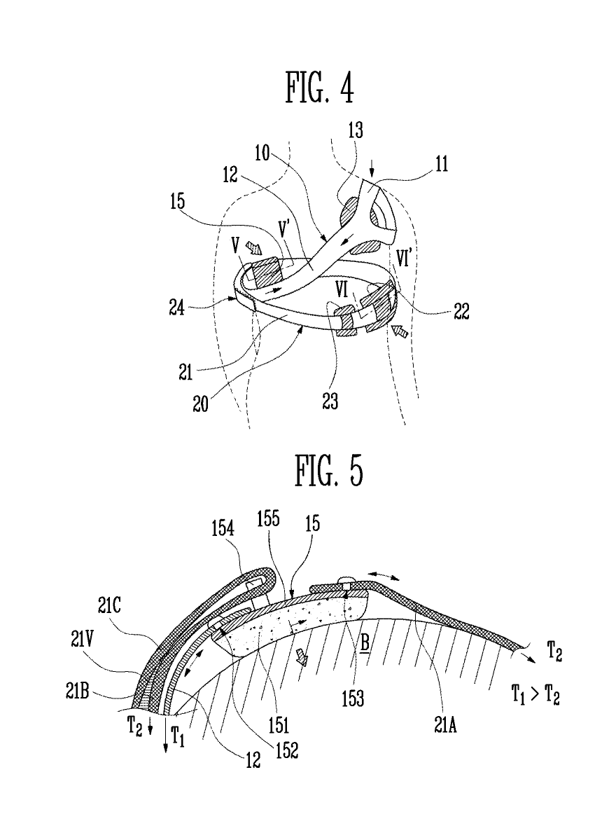

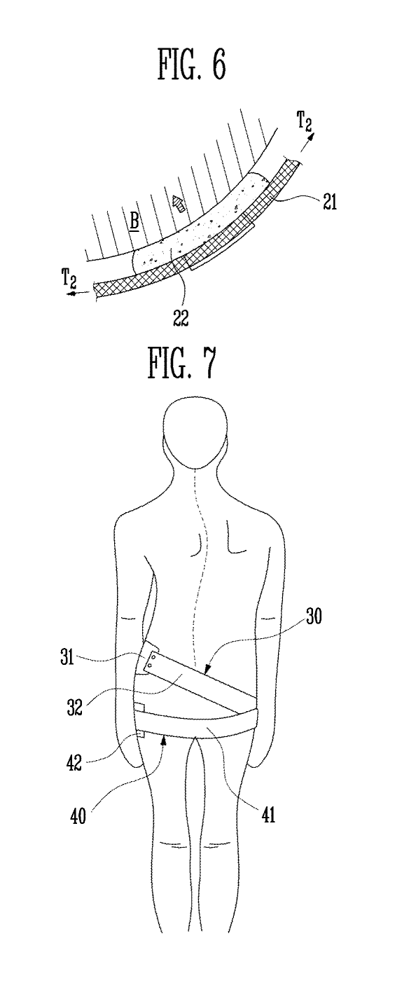

FIG. 5 is a cross-sectional view taken along V-V′ of FIG. 4, and FIG. 6 is a cross-sectional view taken along VI-VI′ of FIG. 4.

First, referring to FIG. 5, the bottom end of the tilt belt 12 is connected to the front costa pad 15 through the length adjustor 152, and a horizontal belt front end 21A is connected to a side opposite thereto. Although the front costa pad 15 and the horizontal belt front end 21A may also be connected through the length adjustor 153, since the length of the horizontal belt may be adjusted by the above-described fixing portion, a necessity thereof is not significant. A cushion member 151 may be disposed at a part of the front costa pad 15 in contact with a body B for even compression and increasing a frictional force. Tension T1 applied to the tilt belt 12 connected to both sides of the front costa pad 15 and tension T2 applied to the horizontal belt 21 may be different, and may have a relationship of, for example, T1>T2. In this case, the overlap support portion such as the front costa pad 15 may be supported to not slide on the body B and may transfer a pressure toward the inside thereof.

Also, a fastening hole 154 formed in, for example, a ring shape may be formed outside a body 155 of the front costa pad 15 and may be fastened to a horizontal belt rear end 21B which wraps around the thorax once. According to the embodiment, the horizontal belt rear end 21B may be held by the fastening hole 154 and oppositely folded, and a folded horizontal belt fixing end 21C may be fixed to the horizontal belt rear end 21B by Velcro 21V provided thereinside. A fastening structure described above is advantageous in terms of easy length adjustment and reinforcement of pressure of the horizontal belt portion 20, but is not limited thereto. Next, referring to FIG. 6, it can be seen that the above-described rear costa pad 22 pressurizes a part of the body B due to the tension T2 of the horizontal belt 21. Here, the rear costa pad 22 may also include a cushion member, and may be installed by allowing the horizontal belt 21 to pass through a ring provided at an outer surface of the rear costa pad 22 such that the rear costa pad 22 may be movable in the longitudinal direction of the horizontal belt 21.

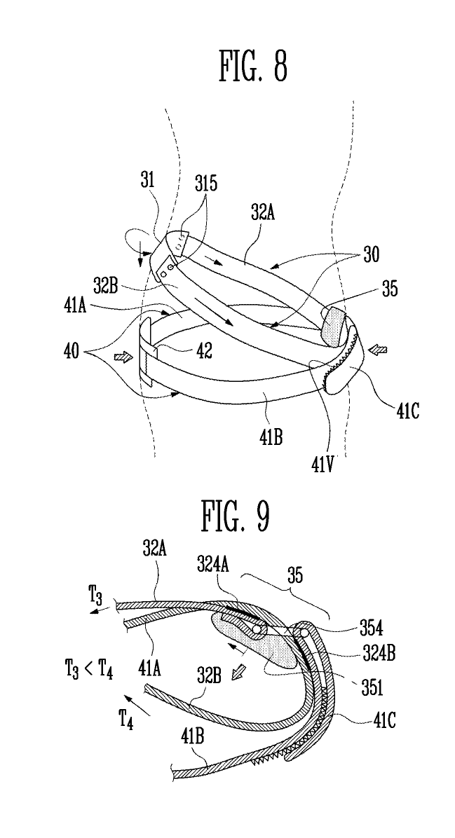

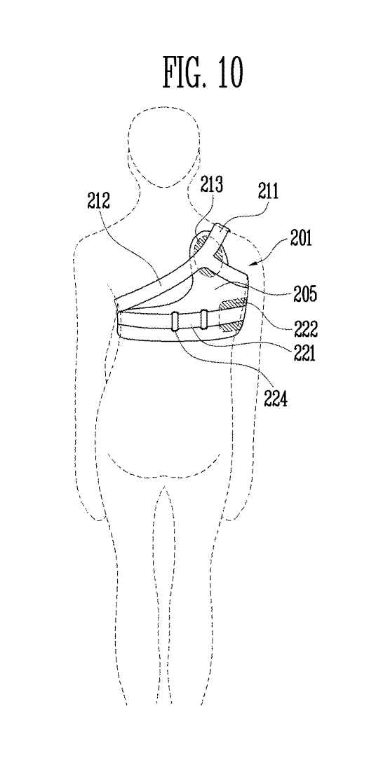

FIG. 7 illustrates a state in which the three-dimensional lower body orthopraxis apparatus according to one embodiment of the present invention is worn, and FIG. 8 is a detailed view illustrating components of the three-dimensional lower body orthopraxis apparatus according to the embodiment of FIG. 7. FIG. 9 is a partially enlarged view illustrating the overlap support portion in FIG. 8.

The three-dimensional lower body orthopraxis apparatus according to the embodiment includes a tilt belt portion 30 which forms one loop shape and a horizontal belt portion 40 which forms another loop shape unlike that of the tilt belt portion 30. A pelvis support portion 31, which surrounds an iliac crest area on one side of the pelvis of the wearer which is relatively raised upward and distorted frontward is provided at a top end of the tilt belt portion 30. The pelvis support portion 31 is one example of the above-described top point support portion and is connected to a tilt belt 32 which obliquely extends downward at a rear side opposite the distortion of the pelvis. The tilt belt 32 is formed of an elastic belt member having elasticity and applies tension downward at the rear side biased to the pelvis support portion 31. A front tilt belt 32A is connected to a front side of the pelvis support portion 31, and a rear tilt belt 32B is connected to a rear thereof, and tensions applied thereto are different. As shown in the drawings, when the tension applied to the rear tilt belt 32B is relatively higher, a force which lowers and rotates the part of the pelvis which is raised upward and distorted frontward in a direction opposite the distortion is applied to the pelvis support portion 31.

A bottom end of the tilt belt 32 is connected to the horizontal belt portion 40, and an overlap support portion 35 which overlaps one part of the horizontal belt portion 40 surrounding the body in a loop shape and pressurizing the body inward and is supported by a force of the pressurization not to slide on the body is formed at a boundary therebetween. The overlap support portion 35 may include, for example, a first cushion pad 351. The first cushion pad 351 is disposed at one side of the front side of the wearer between both a front end of the above-described tilt belt 32A and a rear end of the rear tilt belt 32B and the body of the wearer such that a pressure may be dispersed and a frictional force may be increased. The front costa pad of the above-described three-dimensional upper body orthopraxis apparatus may be a type of first cushion pad. The first cushion pad 351 is pressurized toward the inside of the body and supported by a front end of a front horizontal belt 41A connected to the rear end of the rear tilt belt 32B.

The rear end of the rear tilt belt 32B and the front end of the front horizontal belt 41A may be continuously formed of the same elastic belt member. In this case, since the front end of the front tilt belt 32A is supported by a frictional force which acts on an overlap part 324A thereof, different tensions T3 and T4 may be applied to both sides of the pelvis support portion 31. Meanwhile, an end portion 41C of the rear horizontal belt 41B which wraps around a perimeter of coxae of the wearer from the front end of the front horizontal belt 41A may be held by a ring-shaped fastening hole 354 installed in the front end of the front tilt belt 32A and folded in an opposite direction to be fixed by a fixing portion such as Velcro 41V. This structure may allow a length and tensile strength of the horizontal belt portion 40 which surrounds and pressurizes a coxal part to be adjusted.

Meanwhile, the overlap support portion 35 having such a shape may also perform a function of adjusting a length of the tilt belt portion 30. When the rear tilt belt 32B passes through the above-described ring-shaped fastening hole 354, an outer surface 324B thereof is pressurized by the rear end of the rear horizontal belt 41B such that the length of the rear tilt belt 32B may be maintained in an adjusted state when being worn by the wearer before passing through the ring-shaped fastening hole 354 from the pelvis support portion 31.

Meanwhile, a coxa pad 42 pressurized toward the inside of the body by tension of the horizontal belt 41 may be provided at a position among sections which the horizontal belt portion 40 passes and which corresponds to a coxa below the pelvis support portion 31. The coxa pad 42 may be installed such that its position is movable in a longitudinal direction of the horizontal belt 41. The position of the coxa pad 42 corresponds to a boundary between the front horizontal belt 41A and the rear horizontal belt 41B, and they may actually be continuously connected. The coxa pad 42 corresponds to one example of a second cushion pad disposed opposite the overlap support portion and pressurized toward the inside of the body by the tension of the horizontal belt portion. The rear costa pad of the above-described three-dimensional upper body orthopraxis apparatus also corresponds to the second cushion pad.

The horizontal belt portion 40 pressurizes and realigns coxae, which are loosened by a distortion of the pelvis, toward the inside of the body using the horizontal belt 41 and the coxa pad 42 formed of elastic belt members as described above.

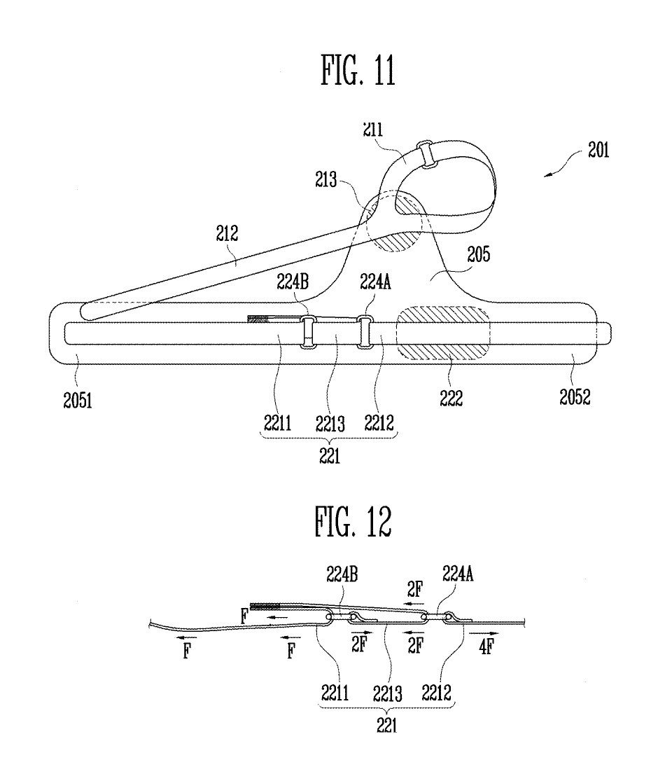

FIG. 10 illustrates a state in which a three-dimensional upper body orthopraxis apparatus according to one embodiment of the present invention is worn, and FIG. 11 is a detailed view illustrating components of the three-dimensional upper body orthopraxis apparatus according to the embodiment of FIG. 10.

A three-dimensional body orthopraxis apparatus 201 according to the embodiment, like the above-described embodiment, includes a top point support portion, a tilt belt portion, and a horizontal belt portion. Also, a flexible skin 205 is additionally included in the dimensional body orthopraxis apparatus 201. The flexible skin 205 is formed to allow a part which overlaps the horizontal belt portion to surround the thorax of the wearer and to allow both ends 2051 and 2052 thereof to be attached to each other. The attachment of both of the ends may be performed using, for example, a Velcro member or may be modified to be a variety of shapes such as a hook, a buckle, and the like as another example. Meanwhile, a part of an outer surface of the flexible skin 205, particularly, a part which overlaps a bottom end of a tilt belt 212 which forms the tilt belt portion or both ends of a horizontal belt 221 which forms the horizontal belt portion, may also be formed using Velcro and the like to allow the tilt belt 212 and the horizontal belt 221 to be pulled in a tilt direction or a horizontal direction and fixed in a state in which tension is applied thereto.

The three-dimensional body orthopraxis apparatus 201 according to the embodiment is for an upper body type apparatus worn on the thorax. The top point support portion includes an annular shoulder band 211 which annularly surrounds a perimeter of one shoulder distorted frontward. The annular shoulder band 211 may be configured to be connected to the tilt belt 212 at a rear side of shoulder. Here, as one example of a top point pressurizing pad, a scapula pad 213 disposed at a position corresponding to the scapula which protrudes rearward and pressurized toward the inside of the body by tensions of the annular shoulder band 211 and the tilt belt 212 may be provided. The scapula pad 213 may be disposed at a top of the flexible skin 205, that is, at a part which extends upward from a part which overlaps the horizontal belt portion.

The flexible skin 205 may be formed of flexible fibers or a mesh material having flexibility and may also be formed of a variety of composite materials to increase elasticity, air permeability, wearability, or the like. The flexible skin 205 defines relative positions of the scapula pad 213 as one example of the above-described top point pressurizing pad, and a rear costa pad 222 as one example of the above-described cushion pad. In other words, the scapula pad 213 and the rear costa pad 222 are fixed to one part of an inside of the flexible skin 205. Accordingly, when a person who wears the three-dimensional body orthopraxis apparatus 201 positions any one of them at a corresponding body part, the other pad may be easily placed. This point increases convenience of the wearer and is helpful for increasing a body orthopraxis effect by allowing accurate wearing of the three-dimensional body orthopraxis apparatus. A slight deviation may be present at each position according to a body size of the wearer, and a size of the apparatus but may be easily adjusted due to material characteristics of the flexible skin 205.

The flexible skin 205 defines relative positions of at least one cushion pad, the tilt belt portion, and the horizontal belt portion described above as well as defines the relative positions of the pads 213 and 222 as described above. The relative positions of the tilt belt portion and the horizontal belt portion may be defined by fixing parts thereof to one position on the flexible skin 205 or supporting at least one part thereof using a fixing structure installed on the flexible skin 205, for example, a structure such as a ring, a hole, or the like which guides a progression direction of a belt.

Meanwhile, the horizontal belt portion may include a cushion pad relatively closer to the scapula pad 213, for example, a front horizontal belt 2212 which extends from the rear costa pad 222 toward a front side of the wearer, and rear horizontal belts 2211 and 2213 which extend toward a rear side of the wearer. The front horizontal belt 2212 and an end portion 2211 of the above-described rear horizontal belts 2211 and 2213, may be formed to be attached to the outer surface of the flexible skin 205. The above-described shape in which the front horizontal belt and the rear horizontal belts are separated may be helpful for realigning a rotational costal malposition by using different tensions thereof applied to the rear costa pad 222.

Also, the horizontal belt 221 at least partially includes an elastic belt member and may include a movable pulley type belt tension structure 224, as shown in the drawing. In the embodiment shown in FIG. 11, a double movable pulley structure in which two pulley rings 224A and 224B which function as movable pulleys are included and an intermediate part 2213 of the horizontal belt 221 is disposed therebetween is applied to the horizontal belt 221. The function and operation principle of movable pulley type belt tension structure 224 will be described below with reference to an additional drawing.

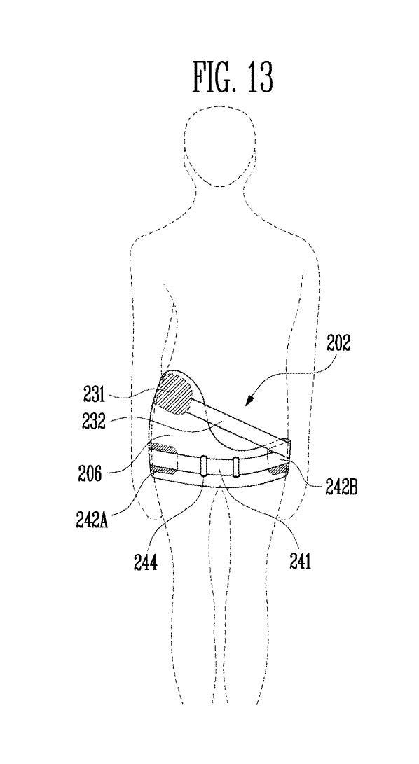

FIG. 12 illustrates the operation principle of the movable pulley type belt tension structure in the embodiment of FIG. 11.

In the embodiment to which the double movable pulley type structure is applied, as described above, the horizontal belt 221 includes the front horizontal belt 2212 and the rear horizontal belt, and the rear horizontal belt includes the end portion 2211 and the intermediate part 2213. A first pulley ring 224A is disposed between an end of the front horizontal belt 2212 and an intermediate part of the intermediate part 2213, and a second pulley ring 224B is disposed between one end of the intermediate part 2213 and an intermediate part of the end portion 2211. Meanwhile, an inner end of the end portion 2211 and an inner end of the intermediate part 2213, which form the rear horizontal belt, are fixed to one position of the above-described flexible skin.

Through the above components, when the wearer pulls the end portion 2211 using a force F, the second pulley ring 224B is pulled by a force 2F in the same direction as that of the force. According to the principle, the first pulley ring 224A is pulled by a force 4F, and thus the above-described rear costa pad 222 of FIG. 11 receives the force 4F in a direction in which the rear horizontal belt is extended. Meanwhile, although the double movable pulley structure is applied to the embodiment, when a single movable pulley structure is applied according to the above-described principle, the force 2F is applied to the above-described rear costa pad 222.

As described above, the movable pulley type belt tension structure maximizes a body orthopraxis effect by allowing the wearer to apply a high tension to the horizontal belt using a small amount of force.

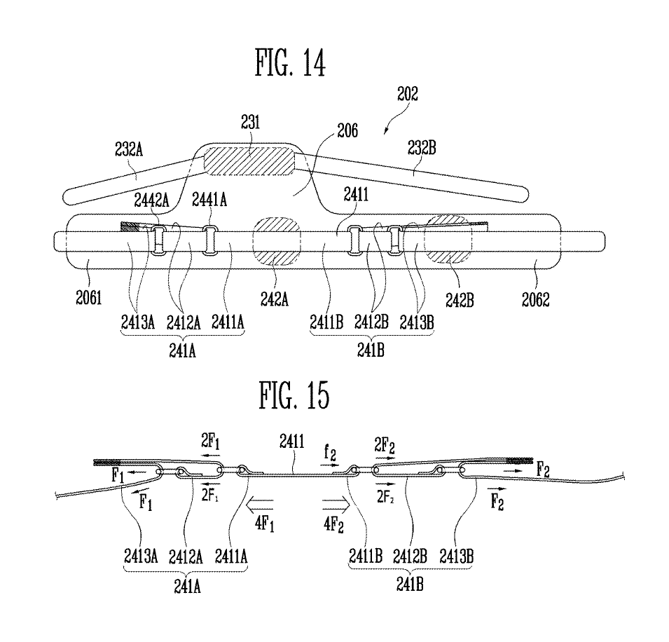

FIG. 13 illustrates a state in which a three-dimensional lower body orthopraxis apparatus according to one embodiment of the present invention is worn, and FIG. 14 is a detailed view illustrating components of the three-dimensional lower body orthopraxis apparatus according to the embodiment of FIG. 13.

A three-dimensional body orthopraxis apparatus 202 according to the embodiment, like the above-described embodiment of FIGS. 10 to 12, includes a top point support portion, a tilt belt portion, a horizontal belt portion, and a flexible skin 206. However, there is a difference such as an upwardly malpositioned pelvic area being supported by a top point support portion. In more detail, in the embodiment, a top point pressurizing pad which forms the top point support portion is a pelvic pad 231 corresponding to an iliac crest area of a side of the pelvis of the wearer which is relatively raised upward. A tilt belt 232, which forms the above-described tilt belt portion and at least partially includes an elastic belt member, may include a front tilt belt 232A which extends from the pelvic pad 231 toward a front side of the wearer and a rear tilt belt 232B which extends toward a rear side of the wearer.

At least one cushion pad disposed to overlap the horizontal belt portion is a pair of coxa pads 242A and 242B, which are fixed to positions corresponding to both sides of a coxa area of the wearer, and is fixed to certain positions on the flexible skin 206 with the pelvic pad 231. Due to this, relative positions thereof are defined. Meanwhile, the flexible skin 206 is formed to allow both ends 2061 and 2062 thereof to be attached to each other while a part which overlaps the horizontal belt portion surrounds the buttocks of the wearer.

Also, the front tilt belt 232A and the rear tilt belt 232B may be formed to be attached to an outer surface of the flexible skin 206. The attachment may be implemented using a member such as Velcro.

Meanwhile, in the three-dimensional body orthopraxis apparatus 202 according to the embodiment, a horizontal belt 241 shown in FIG. 14 is divided into a front horizontal belt 241A which extends from a left coxa pad 242A which is relatively closer to the pelvic pad 231 of the pair of coxa pads 242A and 242B toward the front side of the wearer, and a rear horizontal belt 241B which extends opposite thereto. Like the above-described front tilt belt 232A and rear tilt belt 232B, different tensions are applied to the front belt and the rear belt such that a rotational force may be applied in a direction in which the pelvis of the wearer which is rotationally malpositioned in one direction is restored.

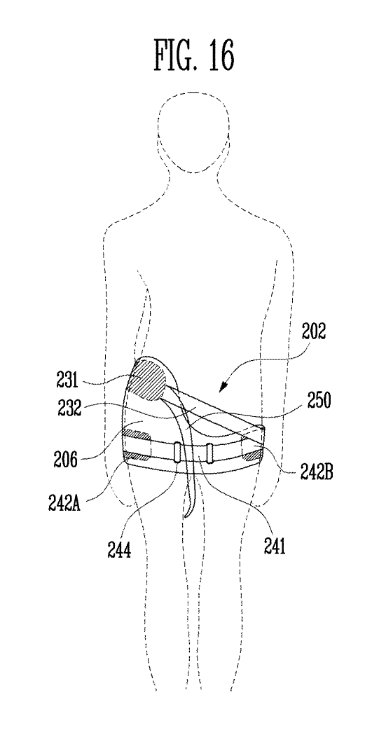

FIG. 15 illustrates an operation principle of a movable pulley type belt tension structure in the embodiment of FIG. 14.

Referring to FIGS. 13 to 15, a movable pulley type belt tension structure 244 may be applied to at least one of the front horizontal belt 241A and the rear horizontal belt 241B which form the above-described horizontal belt 241. In the three-dimensional body orthopraxis apparatus 206 according to the embodiment of FIGS. 14 and 15, a double movable pulley type belt tension structure is applied to each of the front horizontal belt 241A and the rear horizontal belt 241B.

On the basis of a central part 2411 of the horizontal belt 241 overlapping the left coxa pad 242A, two pulley rings 2441A and 2442A are arranged at the front horizontal belt 241A on a left side in the drawing, and an end part 2413A, an intermediate part 2412A, and a central part 2412A of the front horizontal belt 241A are connected via the two pulley rings. Also, an end part 2413B, an intermediate part 2412B, and the central part 2411B are arranged to be connected with two pulley rings at the rear horizontal belt 241B on a right side in the drawing on the basis of the central part 2411.

In the above double movable pulley type belt tension structure, when the wearer pulls the end part 2413A of the front horizontal belt 241A and the end part 2413B of the rear horizontal belt 241B using forces at levels F1 and F2, a force 4F1 acts on a front side of the left coxa pad 242A and a force 4F2 acts on a rear side thereof such that a rotational force may be applied to the pelvis of the wearer by a difference between the forces.

FIG. 16 illustrates a modified example of the embodiment of FIG. 13.

The modified example, in comparison to the embodiment of FIG. 13, has a difference in that an auxiliary belt 250 which passes between the legs of the wearer from one side of the pelvic pad 231, is connected to the other side of the pelvic pad 231, and applies a force pulling the pelvic pad 231 downward is further included in the three-dimensional lower body orthopraxis. Since the other parts are the same, descriptions thereof will be omitted herein.

Meanwhile, a whole body orthopraxis apparatus according to one aspect of the present invention may include a three-dimensional upper body orthopraxis apparatus and a three-dimensional lower body orthopraxis apparatus. The three-dimensional upper body orthopraxis apparatus may be the three-dimensional upper body orthopraxis apparatus according to the embodiment of FIGS. 3 and 4 or the three-dimensional upper body orthopraxis apparatus according to the embodiment of FIGS. 10 and 11. Also, the three-dimensional lower body orthopraxis apparatus may be the three-dimensional lower body orthopraxis apparatus according to the embodiment of FIGS. 7 and 8 or the three-dimensional lower body orthopraxis apparatus according to the embodiment of FIGS. 13 and 14. As described above, the whole body orthopraxis apparatus may be embodied through a variety of combinations of the upper and lower type apparatuses.

Here, the three-dimensional upper orthopraxis apparatus may include an annular shoulder band which annularly surrounds a perimeter of one shoulder of a wearer which is in a state of being rotationally malpositioned frontward rather than a normal state, a first tilt belt portion which at least partially includes an elastic belt, obliquely extends downward from a rear side of the annular shoulder band, and applies tension in a direction opposite the malposition of the one shoulder, and a first horizontal belt portion which at least partially includes an elastic belt, horizontally surrounds and pressurizes the thorax of the wearer, and directly or indirectly supports a bottom end of the first tilt belt portion.

Also, the three-dimensional lower body orthopraxis apparatus may include a pelvis support portion which supports an iliac crest area of a side of the pelvis of the wearer which is raised upward and rotationally malpositioned, a second tilt belt portion which obliquely extends downward from both sides of the pelvis support portion and applies tension in a direction opposite the rotational malposition, and a second horizontal belt portion which at least partially includes an elastic belt, horizontally surrounds and pressurizes the buttocks of the wearer, and directly or indirectly supports a bottom end of the second tilt belt portion.

In the above, the terms “front” and “rear” are used to refer to a front side and a rear side of the wearer's body. Meanwhile, the above-described elastic belt member refers to a strap-shaped member which is generally called a sofa belt or an elastic webbing strap and elastically elongates, but is not limited to the exemplified member. A cushion member which forms a category of pad may also include a variety of forms such as a compressed sponge, a urethane foam, a silicone rubber, and the like.

### INDUSTRIAL APPLICABILITY

The present invention relates to a body orthopraxis apparatus which realigns congenital or acquired body deformities and is applicable to the medial auxiliary apparatus industry.

1. A three-dimensional body orthopraxis apparatus comprising:
a top point support portion which supports a top point which is in a malpositioned state rather than a normal state of both left and right ends of a body;
a tilt belt portion which at least partially comprises an elastic belt, obliquely extends downward from the top point support portion, and applies tension to the top point support portion in a direction opposite a rotational malposition of the top point;
a horizontal belt portion which at least partially comprises an elastic belt and horizontally surrounds and pressurizes the body to partially overlap a bottom end of the tilt belt portion; and
an overlap support portion which forms a boundary at which the bottom end of the tilt belt portion and the horizontal belt portion meet, and is pressurized toward an inside of the body by at least a part of the horizontal belt portion to be supported not to slide on the body,
wherein the top point support portion comprises an annular shoulder band which annularly surrounds a perimeter of one shoulder, and the annular shoulder band is connected to the tilt belt portion at a rear side of the shoulder.

2. The three-dimensional body orthopraxis apparatus of claim 1, wherein the top point support portion further comprises a scapula pad disposed at a position corresponding to a scapula, which protrudes rearward, and pressurized toward the inside of the body by the tensions of the shoulder band and the tilt belt portion.

3. The three-dimensional body orthopraxis apparatus of claim 1, further comprising a costa pad disposed to overlap the horizontal belt portion, disposed at a position corresponding to a protruding costal area, and pressurized toward the inside of the body by the tension of the horizontal belt portion.

4. A three-dimensional body orthopraxis apparatus comprising:
a top point support portion which supports a top point which is in a malpositioned state rather than a normal state of both left and right ends of a body;
a tilt belt portion which at least partially comprises an elastic belt, obliquely extends downward from the top point support portion, and applies tension to the top point support portion in a direction opposite a rotational malposition of the top point;
a horizontal belt portion which at least partially comprises an elastic belt and horizontally surrounds and pressurizes the body to partially overlap a bottom end of the tilt belt portion; and
an overlap support portion which forms a boundary at which the bottom end of the tilt belt portion and the horizontal belt portion meet, and is pressurized toward an inside of the body by at least a part of the horizontal belt portion to be supported not to slide on the body,
wherein the top point support portion comprises a pelvis support portion corresponding to an iliac crest area of one side of the pelvis of a wearer which is relatively raised upward, and further comprises a pair of coxa pads arranged between the horizontal belt portion and the body to correspond to both coxa areas.

5. A three-dimensional body orthopraxis apparatus comprising:
a top point support portion which supports a top point which is in a malpositioned state rather than a normal state of both left and right ends of a body;
a tilt belt portion which at least partially comprises an elastic belt, obliquely extends downward from the top point support portion, and applies tension to the top point support portion in a direction opposite a rotational malposition of the top point;
a horizontal belt portion which at least partially comprises an elastic belt and horizontally surrounds and pressurizes the body;
at least one cushion pad which is pressurized toward an inside of a body by at least a part of the horizontal belt portion, and pressurizes a part of the body; and
a flexible skin which is disposed inside the tilt belt portion and the horizontal belt portion to be in contact with the body of a wearer and defines relative positions of the tilt belt portion, the horizontal belt portion, and the at least one cushion pad.

6. The three-dimensional body orthopraxis apparatus of claim 5, wherein the at least one cushion pad is fixed to one position inside the flexible skin, and at least a part of each of the tilt belt portion and the horizontal belt portion is supported by the flexible skin.

7. The three-dimensional body orthopraxis apparatus of claim 5, wherein an overlap part of the flexible skin which overlaps the horizontal belt portion surrounds the malpositioned body part of the wearer, is formed to allow both ends thereof to be attached to each other, and is formed to allow the tilt belt portion and the horizontal belt portion to be attached to parts of an outer surface of the flexible skin while tension is applied thereto.

8. The three-dimensional body orthopraxis apparatus of claim 5, wherein the top point support portion comprises a top point pressurizing pad which protrudes inward to pressurize a part of the body.

9. The three-dimensional body orthopraxis apparatus of claim 8, wherein the top point pressurizing pad is a pelvic pad corresponding to an iliac crest area of one side of the pelvis of the wearer which is relatively raised upward,
wherein the at least one cushion pad is a pair of coxa pads fixed to positions corresponding to both coxa areas of the wearer,
wherein the tilt belt portion comprises a front tilt belt which extends from the pelvis pad toward a front side of the wearer and a rear tilt belt which extends toward a rear side of the wearer, and
wherein the front tilt belt and the rear tilt belt are attached to the outer surface of the flexible skin.

10. The three-dimensional body orthopraxis apparatus of claim 9, further comprising an auxiliary belt which extends from both sides of the pelvis pad and is supported by the body part of the wearer to apply a force pulling the pelvis pad downward.

11. The three-dimensional body orthopraxis apparatus of claim 5, wherein the top point support portion comprises an annular shoulder band which annularly surrounds a perimeter of one frontwardly distorted shoulder, and the annular should band is connected to the tilt belt portion at a rear side of the shoulder.

12. The three-dimensional body orthopraxis apparatus of claim 11, wherein the top point support portion further comprises a scapula pad disposed at a position corresponding to a scapula, which protrudes rearward, and pressurized toward the inside of the body by the tensions of the annular shoulder band and the tilt belt portion.

13. The three-dimensional body orthopraxis apparatus of claim 5, wherein the horizontal belt portion comprises a front horizontal belt which extends from a cushion pad of the at least one cushion pad which is relatively closer to the top point support portion toward the front side of the wearer, and a rear horizontal belt which extends toward the rear side of the wearer, and
wherein the front horizontal belt and the rear horizontal belt are formed to be attached to an outer surface of the flexible skin.

14. The three-dimensional body orthopraxis apparatus of claim 5, wherein the horizontal belt portion comprises a movable pulley type belt tension structure.

15. A three-dimensional body orthopraxis apparatus comprising:
a three-dimensional upper body orthopraxis apparatus; and
a three-dimensional lower body orthopraxis apparatus,
wherein the three-dimensional upper body orthopraxis apparatus comprises:
an annular shoulder band which annularly surrounds a perimeter of one shoulder which is in a malpositioned state rather than a normal state of shoulders of a wearer;
a first tilt belt portion which at least partially comprises an elastic belt, obliquely extends downward from a rear side of the annular shoulder band, and applies tension in a direction opposite the malposition of the one shoulder; and
a first horizontal belt portion which at least partially comprises an elastic belt, horizontally surrounds and pressurizes the body of the wearer, and directly or indirectly supports a bottom end of the first tilt belt portion, and
wherein the three-dimensional lower body orthopraxis apparatus comprises:
a pelvis support portion which supports an iliac crest area of one side of the pelvis of the wearer which is in a malpositioned state rather than a normal state;
a second tilt belt portion which obliquely extends downward from both sides of the pelvis support portion and applies tension in a direction opposite the rotational malposition; and
a second horizontal belt portion which at least partially comprises an elastic belt, horizontally surrounds and pressurizes the body of the wearer, and directly or indirectly supports a bottom end of the second tilt belt portion.
